(19) United States
(12) Patent Application Publication
(10) Pub. No.: US 2022/0080109 A1
(43) Pub. Date: Mar. 17, 2022

(54) BULB FOR USE WITH IV SET

(71) Applicant: CareFusion 303. Inc., San Diego, CA (US)

(72) Inventors: Leyla Yamin, San Diego, CA (US); Ryan Callahan, Long Beach, CA (US); Kelly Kloster Hon, Del Mar, CA (US)

(21) Appl. No.: 17/018,742

(22) Filed: Sep. 11, 2020

(51) Int. Cl. A61M 5/142; A61M 5/165; A61M 39/10; A61M 5/168

(52) U.S. Cl. CPC A61M 5/1424; A61M 5/165; A61M 25/0097; A61M 5/16831; A61M 39/10

(57) ABSTRACT

Bulbs for use with an IV set are described herein. A bulb includes a bulb body and a filter. The bulb body defines an inlet, an outlet, and a bulb volume in fluid communication with the inlet and the outlet. The filter is in fluid communication with the bulb volume. The filter captures particulate from a flow from the inlet to the outlet. The bulb body is deformable to compress the bulb volume and direct back flow from the bulb volume through the filter, agitating particulate captured in the filter, unclogging the filter and allowing for more flow.

### FIELD OF THE INVENTION

The present disclosure generally relates to intravenous sets, and, in particular, to bulbs for intravenous sets.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag. During operation, medical fluid can be filtered to prevent the transfer of bacteria, microorganisms, and/or other pathogens. In some applications, filters can become clogged, limiting the filtration efficiency and life of the filter.

### SUMMARY

The disclosed subject matter relates to bulbs for use with IV sets. In certain embodiments, a bulb for use with an IV set is disclosed that comprises a bulb body defining an inlet, an outlet, and a bulb volume in fluid communication with the inlet and the outlet; and a filter in fluid communication with the bulb volume, wherein the filter captures particulate from a flow from the inlet to the outlet; wherein the bulb body is deformable to compress the bulb volume and direct back flow from the bulb volume through the filter, agitating particulate captured in the filter

In certain embodiments, a method is disclosed that comprises introducing flow into a bulb volume of a bulb; capturing particulate from the flow into the bulb volume via a filter; deforming the bulb to compress the bulb volume; directing back flow from the bulb volume through the filter; and agitating the particulate captured in the filter.

In certain embodiments, IV sets are disclosed that comprise a first portion of tubing; a second portion of tubing; and a bulb comprising: a bulb body defining an inlet in fluid communication with the first portion of tubing, an outlet in fluid communication with the second portion of tubing, and a bulb volume in fluid communication with the inlet and the outlet; and a filter in fluid communication with the bulb volume, wherein the filter captures particulate from a flow from the inlet to the outlet; wherein the bulb body is deformable to compress the bulb volume and direct back flow from the bulb volume through the filter, agitating particulate captured in the filter.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates a patient receiving an infusion of a medical fluid through an IV pump according to certain aspects of the present disclosure.

FIG. 2 illustrates an IV set according to certain aspects of the present disclosure.

FIG. 3 illustrates an IV set according to certain aspects of the present disclosure.

FIG. 4 illustrates an IV set according to certain aspects of the present disclosure.

### DETAILED DESCRIPTION

The disclosed bulb incorporates the functionality of a hand pump, drip chamber, and filter in a single component. The bulb can be deformable to direct back flow through the filter, allowing particulate embedded in the filter to be displaced. By displacing embedded particulate from the filter, the life of the IV filter can be extended without reducing filtration efficiency. The bulb can be designed to allow increased flow with reduced user fatigue during operation.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

While the following description is directed to the administration of medical fluid using the disclosed bulb, it is to be understood that this description is only an example of usage and does not limit the scope of the claims. Various aspects of the bulb may be used in any application where it is desirable to provide increased fluid flow with reduced user fatigue.

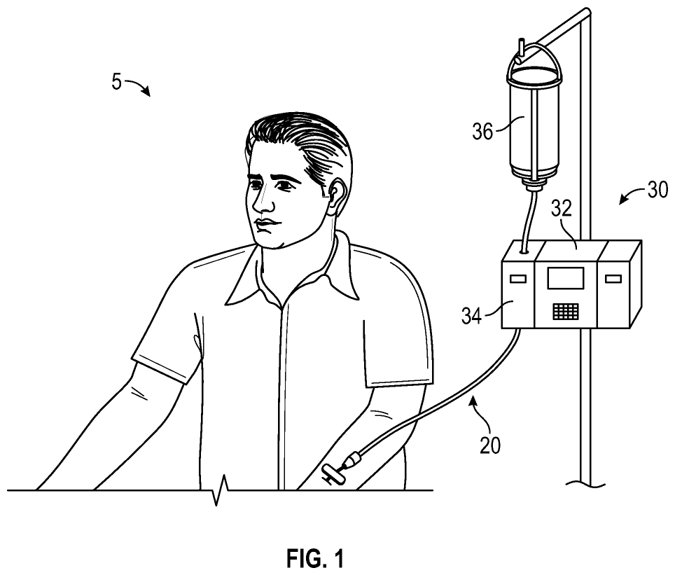

FIG. 1 illustrates a patient 5 receiving an infusion of a medical fluid through an IV pump 30 according to certain aspects of the present disclosure. The IV pump 30 comprises a controller 32 and two pump modules 34. An IV set 20 is connected between a container 36 of the medical fluid and the patient 5. During operation, medical fluid delivered to the patient 5 can be filtered to prevent the transfer of bacteria, microorganisms, and/or other pathogens. During operation, filters can be clogged with particulate, reducing flow through the IV set 20.

In some applications, a clinician can utilize a pump to agitate any particulate trapped within the filter, increasing flow through the filter and the IV set 20.

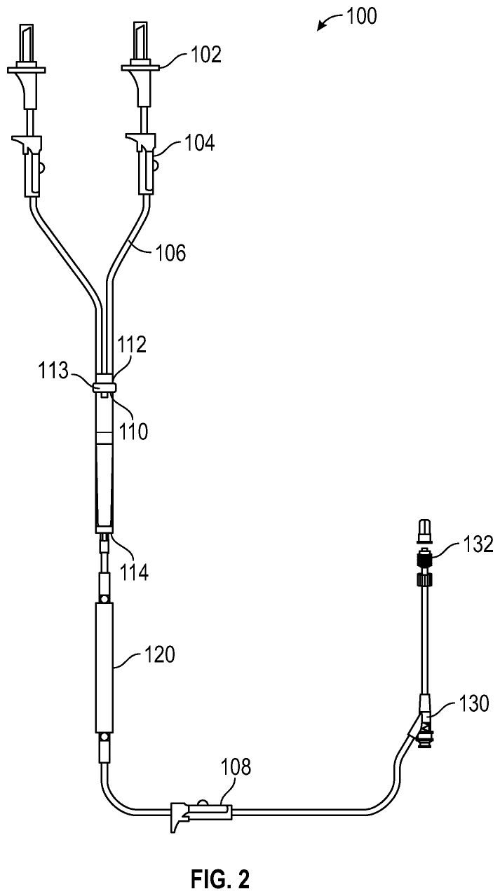

FIG. 2 illustrates an IV set 100 according to certain aspects of the present disclosure. As described herein, the IV set 100 delivers fluid from a fluid source, such as the container 36, to the patient 5 through the tubing 106.

In the depicted example, fluid from the fluid source is introduced into the tubing 106 of the IV set 100. As illustrated, the tubing 106 can be terminated with connectors 102 to facilitate coupling and/or fluid communication with the fluid source. In some embodiments, the connectors 102 can be connector spikes that pierce a membrane of the container 36 to permit fluid communication from the container 36 into the tubing 106. Optionally, the tubing 106 can be coupled to the patient 5 via a port 132.

In some applications, additional medical fluids or treatments can be introduced to the patient via the IV set 100. Optionally, additional medical fluids or treatments can be introduced into the IV set 100 via an injection site 130. Further, clamps 104, 108, can control fluid flow through the tubing 106 of the IV set 100.

In some embodiments, the IV set 100 can include a drip chamber 110 to provide a visual indicator of the flow rate of a medical fluid therethrough. Advantageously, clinicians can monitor and adjust the flow rate of the medical fluid based on the visual indicator provided by the drip chamber 110.

During operation, medical fluid can drip or otherwise flow through the chamber volume of the drip chamber 110. Medical fluid can enter the drip chamber 110 through an upper portion or inlet portion 112 defined in the drip chamber 110. The inlet portion 112 can be in fluid communication with the tubing 106. Fluid flow can exit the drip chamber 110 through a lower portion or outlet portion 114. The outlet portion 114 can be in fluid communication with the tubing 106.

As fluid passes through the drip chamber 110, a clinician can utilize the drip chamber 110 as a visual indicator to observe the dripping or flow of medical fluid therethrough. As can be appreciated the drip chamber 110 can be transparent or semi-transparent.

In some embodiments, the drip chamber 110 can equalize pressure differentials between the chamber volume and the environment during operation. In some embodiments, the drip chamber 110 can be formed from a resilient material to allow the drip chamber 110 to be squeezed or compressed to draw in medical fluid for priming of an IV system.

In the depicted example, the drip chamber 110 can draw in medical fluid for priming of an IV system. As can be appreciated, the drip chamber 110 can be filled with a desired volume of medical fluid during the priming operation.

In some embodiments, the drip chamber 110 can also allow for filtration of medical fluid passing therethrough. As described herein, the drip chamber 110 integrates a filter 113 disposed within the drip chamber 110.

During operation, as medical fluid flows through the drip chamber 110, the fluid can be filtered prior to flowing through tubing 106 and out of the drip chamber 110. In the depicted example, a filter 113 is disposed within the drip chamber 110 to filter fluid passing therethrough. In some embodiments, the filter 113 is disposed within the inlet portion 112 or the outlet portion 114 of the drip chamber 110. Optionally, the filter 113 is disposed within the chamber volume of the drip chamber 110.

As illustrated, fluid within the chamber volume can pass through a filter 113 to prevent the transfer of bacteria, microorganisms, and/or other pathogens to the patient. During operation, fluid can flow through the inlet portion 112 of the drip chamber 110, through a filter 113 to an outlet portion 114 of the drip chamber 110. As can be appreciated, a positive pressure differential can direct fluid flow through the filter 113.

As described herein, the filter 113 can selectively filter the flow through the drip chamber 110. The filter 113 can have an average filter opening ranging between 15 to 200 microns. In some embodiments, the average filter opening can range between 180 to 200 microns. Optionally, the filter 113 can have pores that vary in size. In some embodiments, the filter 113 can be formed from a non-woven filter material.

During operation, the filter 113 can become clogged with particulate, limiting the filtering efficiency and flow through the filter 113. In some applications, the IV set 100 can include a hand pump 120 to dislodge sediment or particulate embedded in the filter 113, extending the life of the filter 113 and increasing the flow through the filter 113. As described herein, the hand pump 120 can direct back flow or back pressure through the filter 113 to dislodge particulate embedded in the filter media. In the depicted example, a clinician can actuate the hand pump 120 to create back flow through the filter 113.

The disclosed bulb overcomes several challenges discovered with respect to certain conventional hand pumps, such as the hand pump 120. One challenge with certain conventional hand pumps is that certain conventional hand pumps can have low flow rates and may be difficult to use for extended periods of time. Because certain conventional hand pumps may cause clinicians fatigue during use, the use of certain conventional hand pumps is undesirable. Further, another challenge with certain conventional IV sets is that certain connectors may include spikes that may inadvertently pierce a fluid container.

Therefore, in accordance with the present disclosure, it is advantageous to provide a bulb as described herein that allows for high flow rates and extended use without clinician fatigue. Further, it is advantageous to provide connectors for IV sets that do not inadvertently pierce fluid containers.

Examples of IV sets that allow for high flow and extended use are now described.

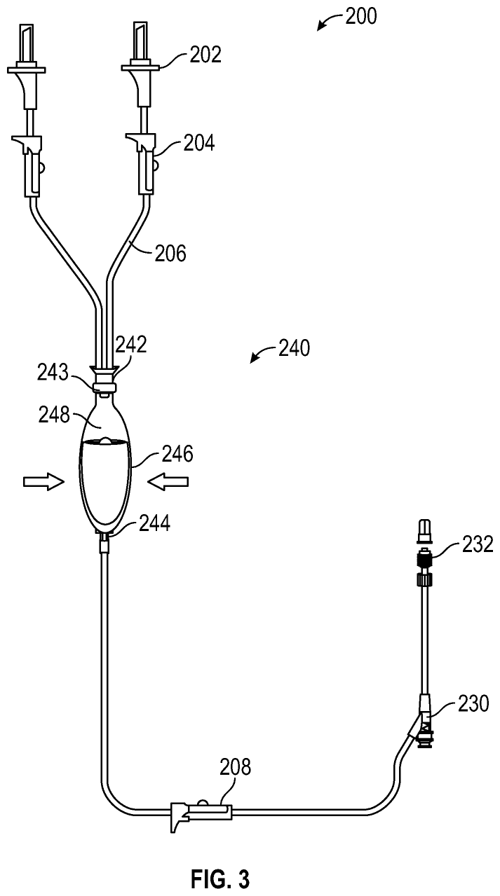

FIG. 3 illustrates an IV set 200 according to certain aspects of the present disclosure. In the depicted example, the IV set 200 can include features that are similar to the IV set 100. Therefore, similar features can be referred to with similar reference numerals. In the depicted example, the IV set 200 utilizes a bulb 240 to provide the functionality of the drip chamber 110 and the hand pump 120 while allowing for increased flow and permitting extended use without clinician fatigue.

In the depicted example, the bulb 240 can provide a visual indicator of the flow rate of a medical fluid therethrough. Advantageously, clinicians can monitor and adjust the flow rate of the medical fluid based on the visual indicator provided by the bulb 240.

During operation, medical fluid can drip or otherwise flow through the bulb volume 248 of the bulb 240. Medical fluid can enter the bulb 240 through an upper portion or inlet portion 242 defined in the bulb body 246. The inlet portion 242 can be in fluid communication with the tubing 206. Fluid flow can exit the bulb volume 248 through a lower portion or outlet portion 244. The outlet portion 244 can be in fluid communication with the tubing 206.

As fluid passes through the bulb volume 248, a clinician can utilize the bulb volume 248 as a visual indicator to observe the dripping or flow of medical fluid therethrough. As can be appreciated the bulb body 246 can be transparent or semi-transparent.

In some embodiments, the bulb 240 can equalize pressure differentials between the bulb volume 248 and the environment during operation. In some embodiments, the bulb body 246 can be formed from a resilient material to allow the bulb body 246 to be squeezed or compressed to draw in medical fluid for priming of an IV system.

In the depicted example, the bulb 240 can draw in medical fluid for priming of an IV system. As can be appreciated, the bulb volume 248 can be filled with a desired volume of medical fluid during the priming operation.

Similar to the drip chamber 110, the bulb 240 can also allow for filtration of medical fluid passing therethrough. As described herein, the bulb 240 integrates a filter 243 disposed within the bulb 240. Advantageously, due to the increased size and volume of the bulb 240, the filter 243 disposed within the bulb 240 can be larger and have more surface area than the filter 243 utilized within the drip chamber 110. As can be appreciated, the filter 243 can be any suitable size.

During operation, as medical fluid flows through the bulb volume 248, the fluid can be filtered prior to flowing out of the bulb 240 and into tubing 206. In the depicted example, a filter 243 is disposed within the bulb 240 to filter fluid passing therethrough. In some embodiments, the filter 243 is disposed within the inlet portion 242 or the outlet portion 244 of the bulb 240. Optionally, the filter 243 is disposed within the bulb volume 248.

As illustrated, fluid within the bulb volume 248 can pass through a filter 243 to prevent the transfer of bacteria, microorganisms, and/or other pathogens to the patient. During operation, fluid can flow through the inlet portion 242 of the bulb 250, through a filter 243 to an outlet portion 244 of the bulb 240. As can be appreciated, a positive pressure differential can direct fluid flow through the filter 243.

As described herein, the filter 243 can selectively filter the flow through the bulb volume 248. The filter 243 can have an average filter opening ranging between 15 to 200 microns. In some embodiments, the average filter opening can range between 180 to 200 microns. Optionally, the filter 243 can have pores that vary in size. In some embodiments, the filter 243 can be formed from a non-woven filter material.

During operation, the filter 243 can become clogged with collected particulate, such as collected clots, small clumps of platelets, or white blood cells, limiting the filtering efficiency and flow through the filter 243. Advantageously, the bulb 240 can be actuated to dislodge sediment or particulate embedded in the filter 243, extending the life of the filter 243 and increasing the flow through the filter 243. As described herein, the bulb 240 can direct back flow or back pressure through the filter 243 to dislodge particulate embedded in the filter media. In the depicted example, a clinician can actuate the bulb 240 to create back flow through the filter 243.

In some embodiments, the bulb body 246 is formed from a resilient or deformable material that allows the bulb volume 248 defined by the bulb body 246 to be reduced upon actuation. The bulb body 246 can be formed from any resilient or deformable material, including, but not limited to silicone, rubber, or thermoplastic elastomers. The bulb body 246 can have a bulbous shape or any other generally ergonomic shape.

By reducing the bulb volume 248, the fluid within the bulb volume 248 is pressurized. Therefore, upon actuation or compression of the bulb 240, back flow is forced through the filter 243, displacing or dislodging particulate from the filter 243. Advantageously, due to the increased bulb volume 248 relative to the hand pump 120, the bulb 240 can improve flow through the IV set 200 rapidly without requiring multiple pumps or actuations. Further, due to the integration of the filter 243 within the bulb 240, the actuation of the bulb 240 can effectively agitated collected particulate within the filter 243, permitting improved flow through the IV set 200.

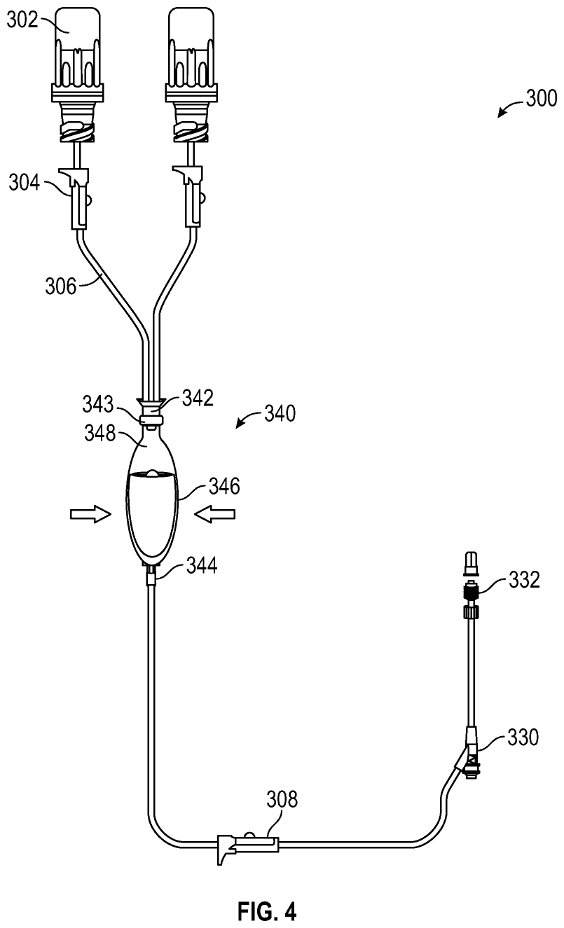

FIG. 4 illustrates an IV set 300 according to certain aspects of the present disclosure. In the depicted example, the IV set 300 can include features that are similar to the IV set 200. Therefore, similar features can be referred to with similar reference numerals. In the depicted example, the IV set 300 utilizes needleless connectors 302 to facilitate coupling and/or fluid communication with the fluid source.

As illustrated, tubing 306 can be terminated with needleless connectors 302 to avoid inadvertent piercing of membranes. In some embodiments, the needleless connectors 302 can include a no-drip feature to prevent leaks or surface contamination. The needleless connectors 302 can further include a luer lock to prevent accidental discharges. Examples of the needleless connectors 302 can include the Texium® connector. As can be appreciated, the needleless connectors 302 can engage with fluid containers that include mating connectors, for example the SmartSite™ connector, the Max Zero connector, and the MaxPlus connector. In some embodiments, the fluid container can allow for pulling aliquots of blood for analysis.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A bulb for use with an IV set, the bulb comprising:
a bulb body defining an inlet, an outlet, and a bulb volume in fluid communication with the inlet and the outlet; and
a filter in fluid communication with the bulb volume, wherein the filter captures particulate from a flow from the inlet to the outlet,
wherein the bulb body is deformable to compress the bulb volume and direct back flow from the bulb volume through the filter, agitating particulate captured in the filter.

2. The bulb of claim 1, wherein the bulb body comprises a resilient material.

3. The bulb of claim 1, wherein the filter is disposed adjacent to the inlet or the outlet.

4. The bulb of claim 1, wherein the filter is disposed within the inlet or the outlet.

5. The bulb of claim 1, wherein the bulb body comprises a transparent material or a semi-transparent material.

6. The bulb of claim 1, wherein the bulb body comprises a generally ovoid shape.

7. A method, comprising:
introducing flow into a bulb volume of a bulb;
capturing particulate from the flow into the bulb volume via a filter;
deforming the bulb to compress the bulb volume;
directing back flow from the bulb volume through the filter; and
agitating the particulate captured in the filter.

8. The method of claim 7, further comprising:
displaying the flow into the bulb volume through a bulb body of the bulb.

9. The method of claim 8, wherein the bulb body comprises a resilient material.

10. The method of claim 7, wherein the bulb comprises a generally ovoid shape.

11. An IV set, comprising:
a first portion of tubing;
a second portion of tubing; and
a bulb comprising:
a bulb body defining an inlet in fluid communication with the first portion of tubing, an outlet in fluid communication with the second portion of tubing, and a bulb volume in fluid communication with the inlet and the outlet; and
a filter in fluid communication with the bulb volume, wherein the filter captures particulate from a flow from the inlet to the outlet,
wherein the bulb body is deformable to compress the bulb volume and direct back flow from the bulb volume through the filter, agitating particulate captured in the filter.

12. The IV set of claim 11, wherein the bulb body comprises a resilient material.

13. The IV set of claim 11, wherein the filter is disposed adjacent to the inlet or the outlet.

14. The IV set of claim 11, wherein the filter is disposed within the inlet or the outlet.

15. The IV set of claim 11, wherein the bulb body comprises a transparent material or a semi-transparent material.

16. The IV set of claim 11, wherein the bulb body comprises a generally ovoid shape.

17. The IV set of claim 11, further comprising a connector in fluid communication with the first portion of tubing.

18. The IV set of claim 17, wherein the connector comprises a needleless connector.

19. The IV set of claim 11, further comprising a clamp coupled to the second portion of tubing.

20. The IV set of claim 11, further comprising an injection site in fluid communication with the second portion of tubing.
